(19) United States
(12) Patent Application Publication
(10) Pub. No.: US 2009/0326443 A1
(43) Pub. Date: Dec. 31, 2009

(54) Preparatory dispensation systems and methods

(75) Inventors: Mahalaxmi Gita Bangera, Renton, WA (US); Edward S. Boyden, Cambridge, MA (US); Roderick A. Hyde, Redmond, WA (US); Muriel Y. Ishikawa, Livermore, CA (US); Eric C. Leuthardt, St. Louis, MO (US); Dennis J. Rivet, Portsmouth, VA (US); Elizabeth A. Sweeney, Seattle, WA (US); Lowell L. Wood, JR., Bellevue, WA (US); Victoria Y. Wood, Livermore, CA (US)

Correspondence Address: SEARETE LLC;CLARENCE T. TEGREENE, 1756 - 114TH AVE., S.E., SUITE 110, BELLEVUE, WA, 98004, US

(73) Assignee: Searete LLC, a limited Liability corporation of the State of Delaware

(21) Appl. No.: 12/217,134

(22) Filed: Jun. 30, 2008

(51) Int. Cl. A61M 31/00

(52) U.S. Cl. 604/65

(57) ABSTRACT

Methods and systems are described for receiving wireless control signal content into a module containing one or more active ingredients and causing one or more dispensations from the module according to a programmatic dispensation profile partly dependent on the wireless control signal content and partly dependent on an attribute of the module; or for obtaining data indicating a future event time intended by an individual, and causing a bioactive material administration to the individual in response to the data to occur about or before the future event time.

### SUMMARY

In one aspect, a method includes but is not limited to receiving wireless control signal content into a module containing one or more active ingredients and causing one or more dispensations from the module according to a programmatic dispensation profile partly dependent on the wireless control signal content and partly dependent on an attribute of the module. In addition to the foregoing, other method aspects are described in the claims, drawings, and text forming a part of the present disclosure.

In one or more various aspects, related systems include but are not limited to circuitry and/or programming for effecting the herein referenced method aspects; the circuitry and/or programming can be virtually any combination of hardware, software, and/or firmware configured to effect the herein referenced method aspects depending upon the design choices of the system designer.

In one aspect, a system includes but is not limited to circuitry receiving wireless control signal content into a module containing one or more active ingredients and causing one or more dispensations from the module according to a programmatic dispensation profile partly dependent on the wireless control signal content and partly dependent on an attribute of the module. In addition to the foregoing, other system aspects are described in the claims, drawings, and text forming a part of the present disclosure.

In one aspect, a method includes but is not limited to obtaining data indicating a future event time intended by an individual and causing a bioactive material administration to the individual in response to the data to occur about or before the future event time. In addition to the foregoing, other method aspects are described in the claims, drawings, and text forming a part of the present disclosure.

In one or more various aspects, related systems include but are not limited to circuitry and/or programming for effecting the herein referenced method aspects; the circuitry and/or programming can be virtually any combination of hardware, software, and/or firmware configured to effect the herein referenced method aspects depending upon the design choices of the system designer.

In one aspect, a system includes but is not limited to circuitry for obtaining data indicating a future event time intended by an individual and causing a bioactive material administration to the individual in response to the data to occur about or before the future event time. In addition to the foregoing, other system aspects are described in the claims, drawings, and text forming a part of the present disclosure.

In addition to the foregoing, various other method and/or system and/or program product aspects are set forth and described in the teachings such as text (e.g., claims and/or detailed description) and/or drawings of the present disclosure.

The foregoing is a summary and thus may contain simplifications, generalizations, inclusions, and/or omissions of detail; consequently, those skilled in the art will appreciate that the summary is illustrative only and is NOT intended to be in any way limiting. Other aspects, features, and advantages of the devices and/or processes and/or other subject matter described herein will become apparent in the teachings set forth herein.

In one or more various aspects, related systems include but are not limited to circuitry and/or programming for effecting herein-referenced method aspects; the circuitry and/or programming can be virtually any combination of hardware, software, and/or firmware configured to effect the herein-referenced method aspects depending upon the design choices of the system designer. In addition to the foregoing, various other method and/or system aspects are set forth and described in the teachings such as text (e.g., claims and/or detailed description) and/or drawings of the present disclosure.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

FIG. 1 depicts exemplary environments in which one or more technologies may be implemented.

FIG. 2 depicts a high-level logic flow of an operational process.

FIG. 3 depicts exemplary environments in which one or more technologies may be implemented.

FIG. 4 depicts a high-level logic flow of an operational process.

FIGS. 5-15 depict exemplary environments in which one or more technologies may be implemented.

FIGS. 16-17 depict variants of the flow of FIG. 2.

FIGS. 18-19 depict variants of the flow of FIG. 4.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

Those having skill in the art will recognize that the state of the art has progressed to the point where there is little distinction left between hardware, software, and/or firmware implementations of aspects of systems; the use of hardware, software, and/or firmware is generally (but not always, in that in certain contexts the choice between hardware and software can become significant) a design choice representing cost vs. efficiency tradeoffs. Those having skill in the art will appreciate that there are various vehicles by which processes and/or systems and/or other technologies described herein can be effected (e.g., hardware, software, and/or firmware), and that the preferred vehicle will vary with the context in which the processes and/or systems and/or other technologies are deployed. For example, if an implementer determines that speed and accuracy are paramount, the implementer may opt for a mainly hardware and/or firmware vehicle; alternatively, if flexibility is paramount, the implementer may opt for a mainly software implementation; or, yet again alternatively, the implementer may opt for some combination of hardware, software, and/or firmware. Hence, there are several possible vehicles by which the processes and/or devices and/or other technologies described herein may be effected, none of which is inherently superior to the other in that any vehicle to be utilized is a choice dependent upon the context in which the vehicle will be deployed and the specific concerns (e.g., speed, flexibility, or predictability) of the implementer, any of which may vary. Those skilled in the art will recognize that optical aspects of implementations will typically employ optically-oriented hardware, software, and or firmware.

In some implementations described herein, logic and similar implementations may include software or other control structures suitable to operation. Electronic circuitry, for example, may manifest one or more paths of electrical current constructed and arranged to implement various logic functions as described herein. In some implementations, one or more media are configured to bear a device-detectable implementation if such media hold or transmit a special-purpose device instruction set operable to perform as described herein. In some variants, for example, this may manifest as an update or other modification of existing software or firmware, or of gate arrays or other programmable hardware, such as by performing a reception of or a transmission of one or more instructions in relation to one or more operations described herein. Alternatively or additionally, in some variants, an implementation may include special-purpose hardware, software, firmware components, and/or general-purpose components executing or otherwise invoking special-purpose components. Specifications or other implementations may be transmitted by one or more instances of tangible transmission media as described herein, optionally by packet transmission or otherwise by passing through distributed media at various times.

Alternatively or additionally, implementations may include executing a special-purpose instruction sequence or otherwise invoking circuitry for enabling, triggering, coordinating, requesting, or otherwise causing one or more occurrences of any functional operations described above. In some variants, operational or other logical descriptions herein may be expressed directly as source code and compiled or otherwise invoked as an executable instruction sequence. In some contexts, for example, C++ or other code sequences can be compiled directly or otherwise implemented in high-level descriptor languages (e.g., a logic-synthesizable language, a hardware description language, a hardware design simulation, and/or other such similar mode(s) of expression). Alternatively or additionally, some or all of the logical expression may be manifested as a Verilog-type hardware description or other circuitry model before physical implementation in hardware, especially for basic operations or timing-critical applications. Those skilled in the art will recognize how to obtain, configure, and optimize suitable transmission or computational elements, material supplies, actuators, or other common structures in light of these teachings.

In a general sense, those skilled in the art will recognize that the various embodiments described herein can be implemented, individually and/or collectively, by various types of electro-mechanical systems having a wide range of electrical components such as hardware, software, firmware, and/or virtually any combination thereof, and a wide range of components that may impart mechanical force or motion such as rigid bodies, spring or torsional bodies, hydraulics, electro-magnetically actuated devices, and/or virtually any combination thereof. Consequently, as used herein “electro-mechanical system” includes, but is not limited to, electrical circuitry operably coupled with a transducer (e.g., an actuator, a motor, a piezoelectric crystal, a Micro Electro Mechanical System (MEMS), etc.), electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of memory (e.g., random access, flash, read only, etc.)), electrical circuitry forming a communications device (e.g., a modem, communications switch, optical-electrical equipment, etc.), and/or any non-electrical analog thereto, such as optical or other analogs. Those skilled in the art will also appreciate that examples of electro-mechanical systems include but are not limited to a variety of consumer electronics systems, medical devices, as well as other systems such as motorized transport systems, factory automation systems, security systems, and/or communication/computing systems. Those skilled in the art will recognize that electro-mechanical as used herein is not necessarily limited to a system that has both electrical and mechanical actuation except as context may dictate otherwise.

In a general sense, those skilled in the art will also recognize that the various aspects described herein which can be implemented, individually and/or collectively, by a wide range of hardware, software, firmware, and/or any combination thereof can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of memory (e.g., random access, flash, read only, etc.)), and/or electrical circuitry forming a communications device (e.g., a modem, communications switch, optical-electrical equipment, etc.). Those having skill in the art will recognize that the subject matter described herein may be implemented in an analog or digital fashion or some combination thereof.

Those skilled in the art will further recognize that at least a portion of the devices and/or processes described herein can be integrated into an image processing system. A typical image processing system may generally include one or more of a system unit housing, a video display device, memory such as volatile or non-volatile memory, processors such as microprocessors or digital signal processors, computational entities such as operating systems, drivers, applications programs, one or more interaction devices (e.g., a touch pad, a touch screen, an antenna, etc.), control systems including feedback loops and control motors (e.g., feedback for sensing lens position and/or velocity; control motors for moving/distorting lenses to give desired focuses). An image processing system may be implemented utilizing suitable commercially available components, such as those typically found in digital still systems and/or digital motion systems.

Those skilled in the art will likewise recognize that at least some of the devices and/or processes described herein can be integrated into a data processing system. Those having skill in the art will recognize that a data processing system generally includes one or more of a system unit housing, a video display device, memory such as volatile or non-volatile memory, processors such as microprocessors or digital signal processors, computational entities such as operating systems, drivers, graphical user interfaces, and applications programs, one or more interaction devices (e.g., a touch pad, a touch screen, an antenna, etc.), and/or control systems including feedback loops and control motors (e.g., feedback for sensing position and/or velocity; control motors for moving and/or adjusting components and/or quantities). A data processing system may be implemented utilizing suitable commercially available components, such as those typically found in data computing/communication and/or network computing/communication systems.

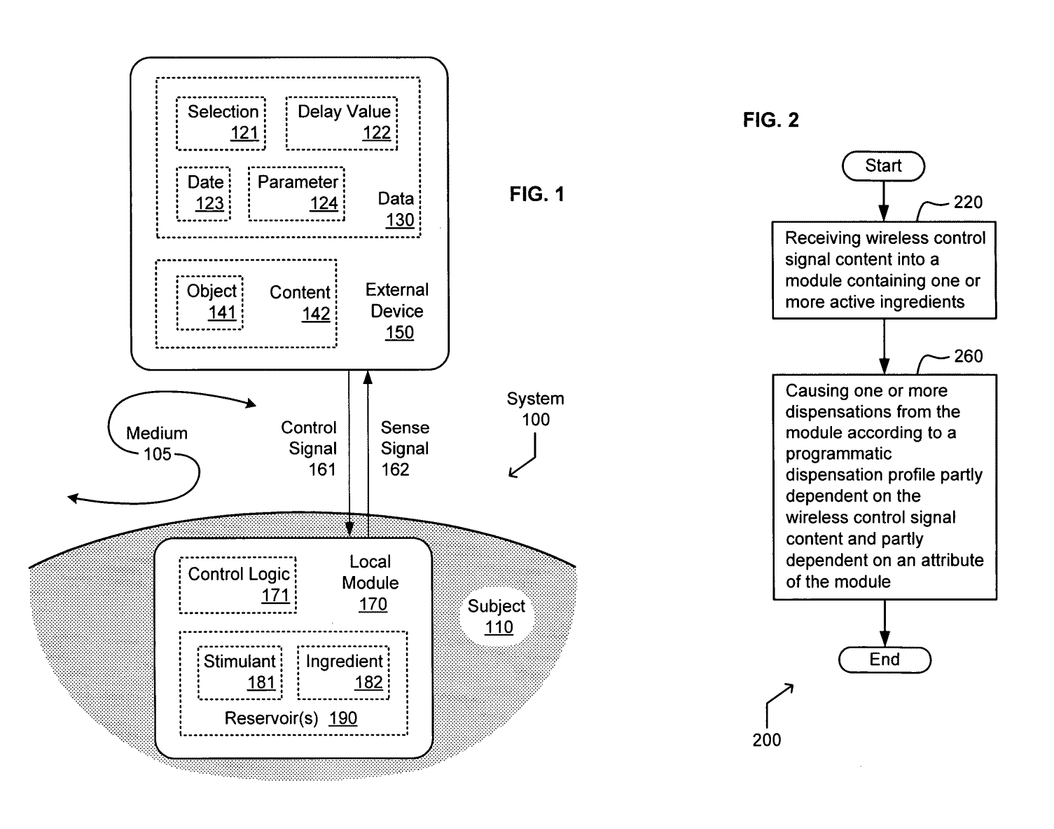

With reference now to FIG. 1, shown is a system 100 comprising one or more local modules 170 configured to handle one or more control signals 161 or sense signals 162, interacting with one or more external devices 150 across at least one wireless medium 105. This may include receiving one or more selections 121, delay values 122, dates 123, operational parameters 124, or other such data 130 as described below. In some variants, for example, such data objects 141 or other content 142 are received and acted upon by control logic 171, such as for controlling how a stimulant 181 or other ingredients 182 are dispensed (from one or more reservoirs 190, e.g.) locally to a mammal or other subject 110.

With reference now to FIG. 2, shown is a flow 200 comprising operation 220—receiving wireless control signal content into a module containing one or more active ingredients (e.g. an optical sensor, antenna, or other such wireless-signal-receiving portion of control logic 171 receiving one or more control signals 161 via a free space medium 105). This can occur, for example, in a context in which module 170 contains one or more active-ingredient-containing reservoirs suitable for facilitating an administration to or other dispensation to a subject 110. In some variants, for example, such reservoirs may include one or more stimulants 181 or other such active ingredients, optionally in a mixture or other compound comprising a pharmaceutically acceptable carrier. Alternatively or additionally, such local modules 170 may include an adhesive patch suitable for placement onto a subject's body surface, an injectable or ingestible conduit or capsule, a handheld inhaler or other portable dispensing device, or otherwise in, on, or near a subject of interest.

Flow 200 further comprises operation 260—causing one or more dispensations from the module according to a programmatic dispensation profile partly dependent on the wireless control signal content and partly dependent on an attribute of the module (e.g. control logic 171 adapting and/or implementing one or more dates 123 and/or other parameters 124 effectively controlling which, where, how, and/or when one or more available reservoirs 190 are to dispense a stimulant 181 and/or other available ingredients 182). This can occur, for example, in a context in which one or more instances of control logic 171 effectively generate the programmatic dispensation profile to reflect at least (a) a type and/or status of the ingredient(s) 182 and/or reservoirs 190 and (b) a portion of the content 142 received as control signal 161. In some variants, for example, control logic 171 responds by indicating some or all of the programmatic dispensation profile as sense signal 162, such as for indicating any tasks that module 170 is (or is not) able to complete. Alternatively or additionally, one or more external devices 150 may generate such a programmatic dispensation profile as data 130 informed by a type and/or status of module 170, for example, optionally transmitted as additional content of control signal 161.

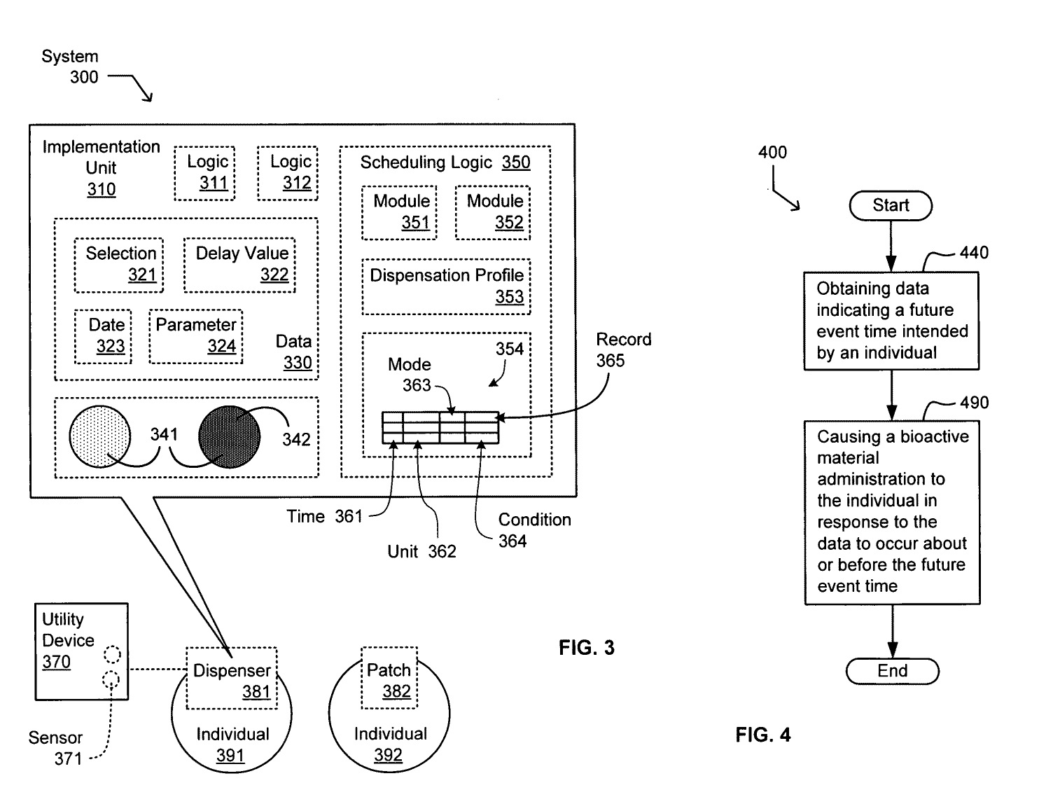

With reference now to FIG. 3, shown is a system 300 comprising one or more implementation units 310 such as may be included (on a chip, e.g.) in an adhesive patch, for example, suitable for bioactive material administration through skin or mucous membranes. Such implementation units may likewise reside in an ingestible capsule, a fluid-bearing conduit, or in other dispensers 381 for administering bioactive materials as exemplified herein. In some variants, for example, implementation unit 310 may include one or more instances of pattern recognition logic 311; invocation logic 312; selections 321, delay values 322, dates 323, or other parameters 324 of data 330; bioactive materials 341, 342 in solid or other form conducive to controllable administration to a subject individual; or scheduling logic 350. Such scheduling logic 350 may, for example, include one or more modules 351, 352 operable to configure or implement one or more records 365 or other elements of a dispensation profile 353, 354. Such records may identify one or more instances of initiation or other times 361, dispensers or other units 362, controllable dosages or other such modes 363 of administration, or conditions 364 enabling or triggering administrations or notifications. In some variants, one or more sensors 371 or other implementation components may reside in a handheld appliance or other such utility device 370 configured to guide or otherwise interact with a patch 382 or other dispenser 381 operable for administering bioactive material to one or more individuals 391, 392.

With reference now to FIG. 4, shown is a flow 400 comprising operation 440—obtaining data indicating a future event time intended by an individual (e.g. module 351 of scheduling logic 350 receiving or implementing one or more selections 321, delay values 322, dates 323, or other such parameters 324 suggesting an expectation, a hope, a behavior pattern, or other such intentions of one or more individuals 391 to whom a material might be administered). This can occur, for example, in a context in which a transdermal patch 382 includes or responds to an implementation unit 310 configured to obtain such data 330—optionally via one or more interfaces, timers, portable items, or other such utility devices 370 having user input elements or other sensors 371. In some variants, for example, a subject individual 391 can express such intention by selecting an event from a menu, by referring to the event in a natural language expression, or in some other way discernable by pattern recognition logic 311. Alternatively or additionally, pattern recognition logic 311 may be configured to extract such data 330 by similar observations of a meeting participant or other individual with whom the subject interacts, optionally with a confirmatory interaction with the subject.

Flow 400 further comprises operation 490—causing a bioactive material administration to the individual in response to the data to occur about or before the future event time (e.g. module 352 of scheduling logic 350 generating or adapting one or more records 365 indicative of a future administration at a time 361 before or within a few hours of the future event time determined as data 330). This can occur, for example, in a context in which scheduling logic 350 performs operation 490 by implementing the administration, such as (a) by triggering an immediate administration after waiting an amount of time commensurate with delay value 322, (b) transmitting such a delay value or other parameter 324 relating to a suitable delay to be implemented by a smart dispenser, and/or (c) by generating a delay value 322 suitable for causing the administration at the proper time determined in response to the type and/or configuration of the dispenser(s) to be used. Alternatively or additionally, module 352 may comprise primary circuitry for causing the bioactive material administration, in an embodiment in which invocation logic 312 comprises secondary circuitry for invoking the primary circuitry. In some variants, moreover, some or all logic of implementation unit 310 may reside within utility device 370 and/or may interact with one or more other dispensers 381 for administering one or more bioactive materials 341, 342 to another individual 392, for example, to support that individual's intention.

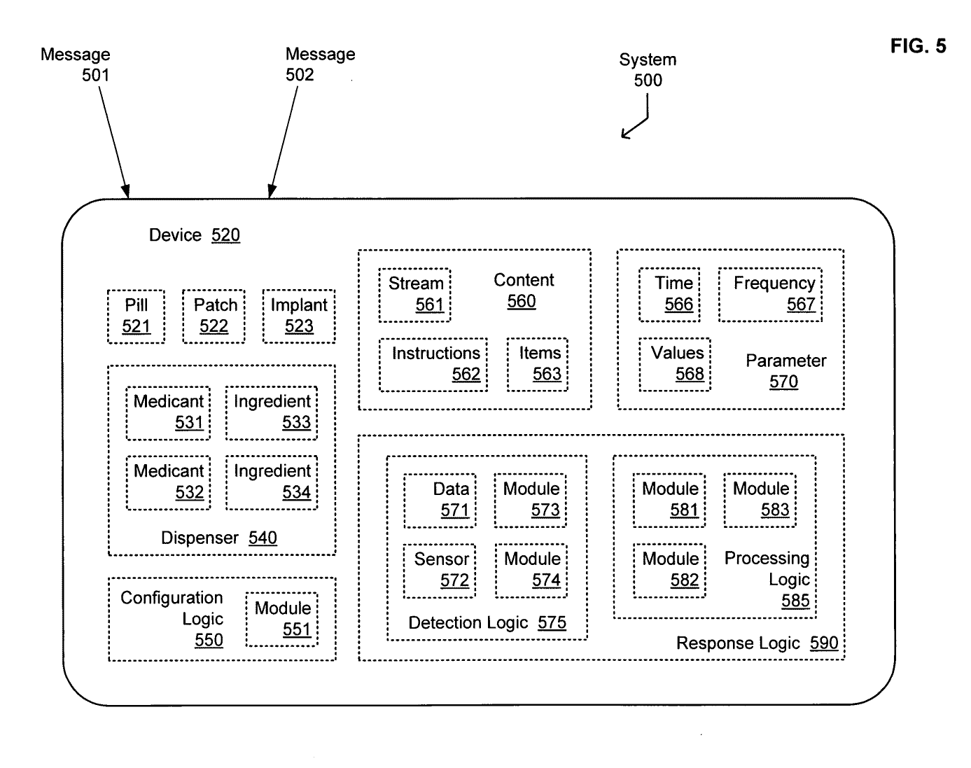

With reference now to FIG. 5, shown is a system 500 comprising a pill 521, patch 522, transvascular conduit, implant 523, or other such device 520 suitable for implementing one or more dispensers 540 for medicants 531, 532 and/or other ingredients 533, 534, for example, as guided by various sensors 572 or other modules 551, 573, 574, 581, 582, 583 of configuration logic 550 and/or response logic 590. Such logic may, for example, generate or use one or more instances of data streams 561, instructions 562, configuration items 563, or other such content 560; times 566, frequencies 567, threshold adjustment values 568, or other such parameters 570; messages 501, 502; or other data 571.

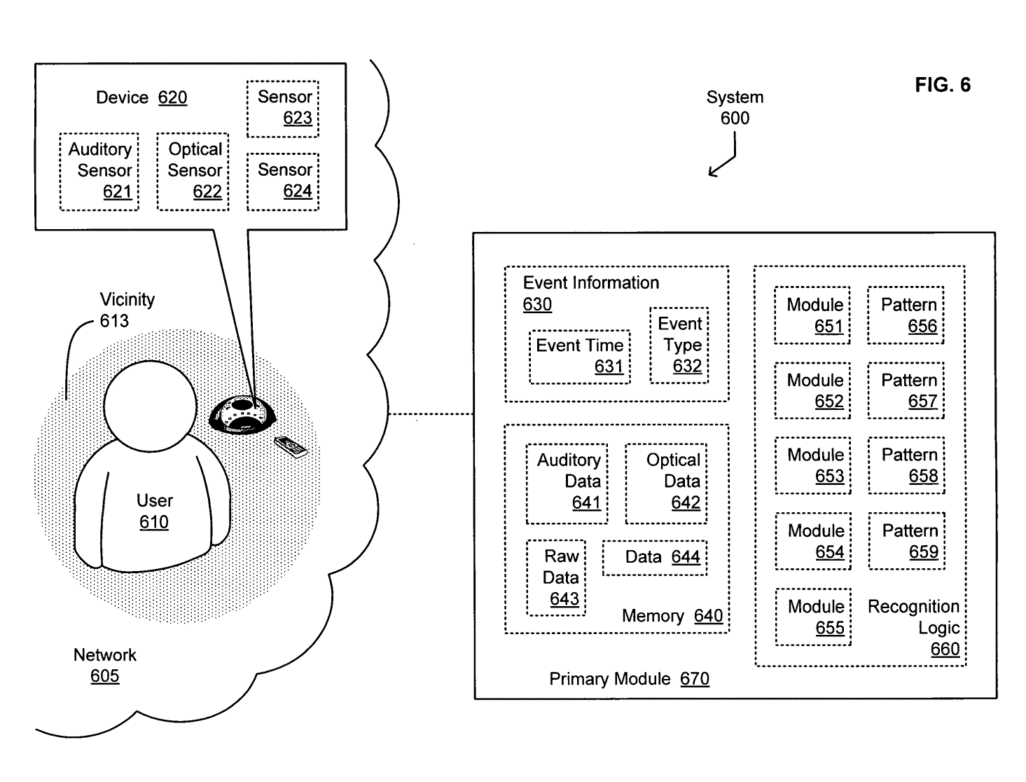

With reference now to FIG. 6, shown is a system 600 comprising primary module 670 configured to obtain one or more instances of event times 631, event types 632, or other such event information 630 intended by a user 610. In some variants, for example, one or more auditory sensors 621, optical sensors 622, or other sensors 623, 624 reside in a vicinity 613 of user 610 and/or in an interface or other device 620 of a network 605 from which primary module 670 can receive a signal. Such signals may contain auditory data 641, optical data 642, raw data 643, or other data 644 held in memory 640, for example. Primary module 670 may likewise include one or more modules 651, 652, 653, 654, 655 of recognition logic 660 configured to recognize words, shapes, actions, or other such patterns 656, 657, 658, 659 signifying user selections or other manifestations of future event information.

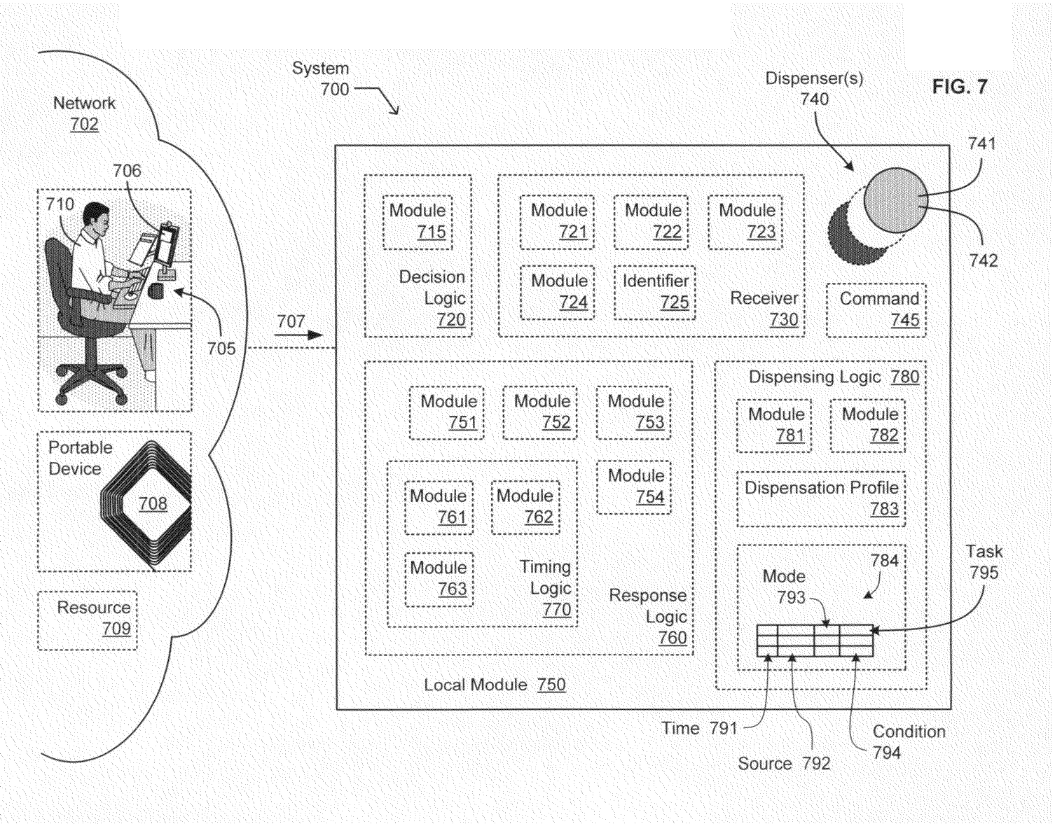

With reference now to FIG. 7, shown is a system 700 comprising one or more local modules 750 operable to communicate with one or more individuals 710 or other resources 709 in network 702. (Such modules may, in some contexts, be “local” to a dispensation recipient presently or at a future time closer to a scheduled event.) Such communication may include output 706 and/or input 707 vis-à-vis interface 705 or a portable device 708, for example. In some embodiments, local module 750 may include one or more instances of modules 715, 721, 722, 723, 724 of decision logic 720 or receivers 730; one or more identifiers 725 and/or commands 745; modules 751, 752, 753, 754, 761, 762, 763 of timing logic 770 or other response logic 760; or modules 781, 782 of dispensing logic 780. Such logic may indicate one or more event times 791; sources 792 of therapeutic or other ingredients 741, 742; delivery modes 793; performance conditions 794; or other attributes of dispensation profiles 783, 784 or other tasks 795 to be performed by dispensers 740 or other entities.

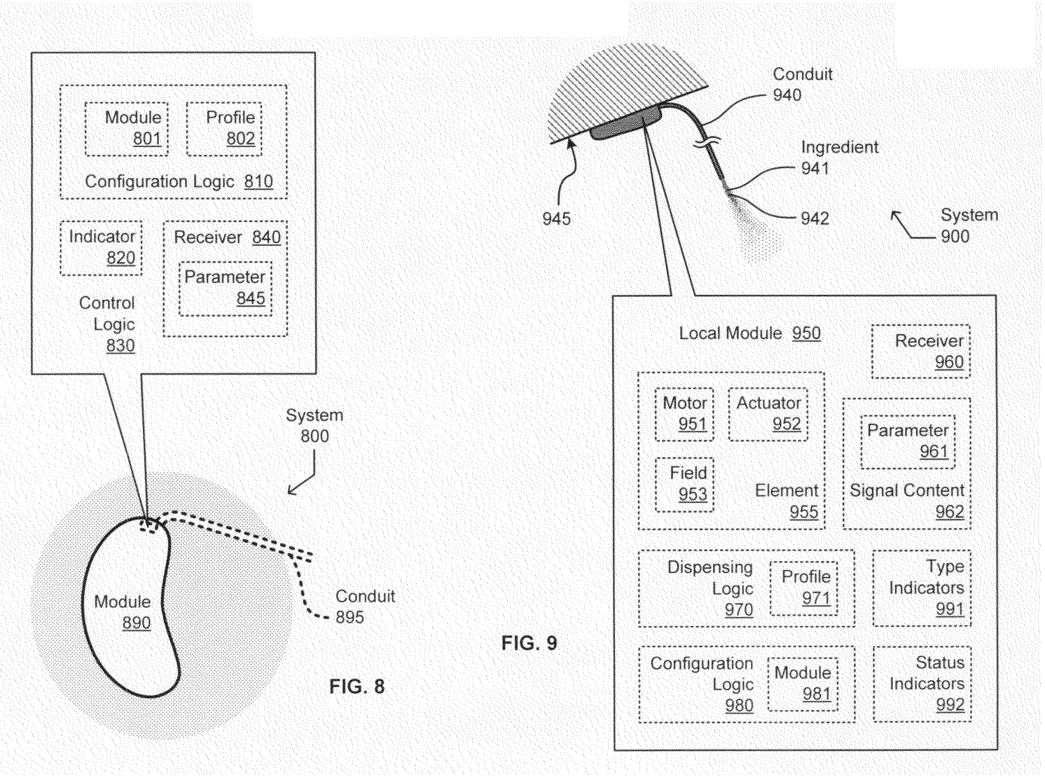

With reference now to FIG. 8, shown is a system 800 comprising a dispenser or other module 890 configured to include control logic 830. Such logic may include one or more instances of receivers 840 operable to handle subject or module status indicators 820; user inputs, event times, or other control parameters 845; modules 801 of configuration logic 810 operable for adapting or implementing profiles 802; or various response logic as described herein. In some variants, for example, module 890 may include or otherwise provide material via one or more in-dwelling or other conduits 895.

With reference now to FIG. 9, shown is a system 900 comprising a local module 950 operable to dispense one or more ingredients 941, 942 via one or more conduits 940. In some variants, for example, local module 950 be positioned on or otherwise configured to interact with skin, sinus passages, stomach lining, or other such membranes 945 of a subject's body. In some variants, for example, local module 950 may facilitate such dispensations via one or more instances of motors 951, actuators 952, electrical or magnetic fields 953, or other such dispensation elements 955. Alternatively or additionally, local module 950 may include one or more receivers 960, optionally configured to extract one or more parameters 961 from radio frequency or other signal content 962 passing through a wireless medium. In some variants, local module 950 may include one or more instances of dispensing logic 970 configured to implement one or more profiles 971, one or more modules 981 of configuration logic 980 operable to transmit and/or respond to type indicators 991 and/or status indicators 992 as described herein.

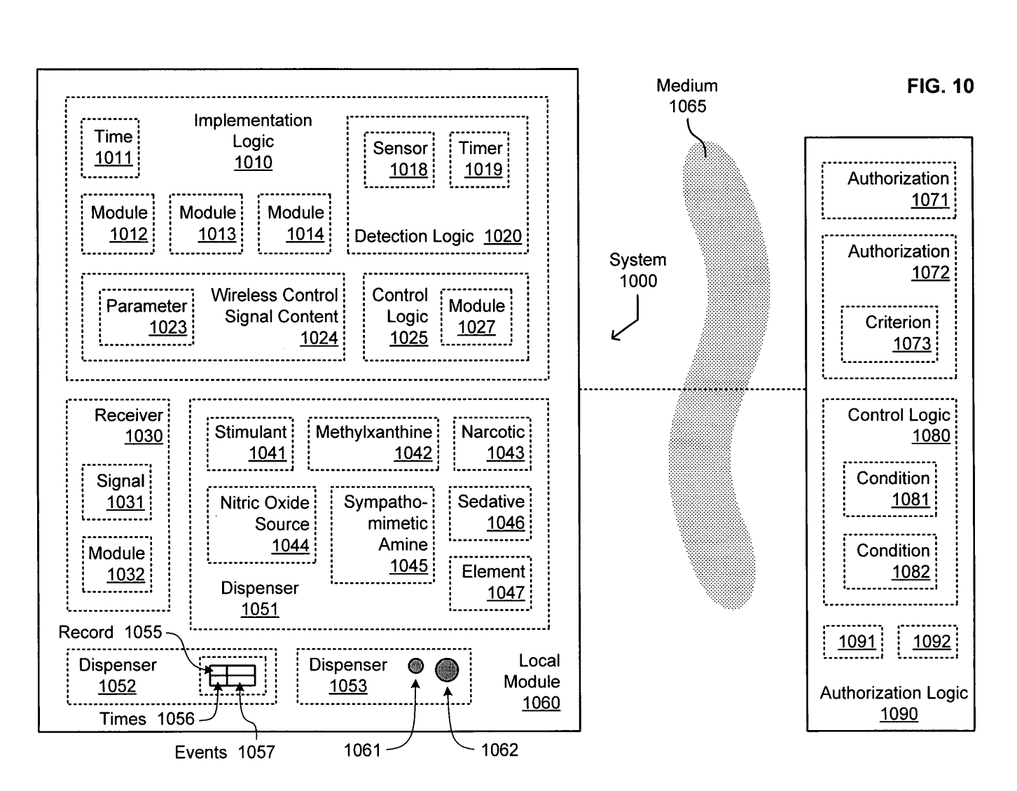

With reference now to FIG. 10, shown is a system 1000 comprising a local module 1060 including one or more instances of implementation logic 1010, receivers 1030, or dispensers 1051, 1052, 1053. As shown, local module 1060 is configured to receive data from and/or transmit data to one or more instances of authorization logic 1090 across at least one wireless medium 1065. In some contexts, for example, local module 1060 may receive one or more authorizations 1071, 1072 (optionally contingent on criteria 1073, for example) from control logic 1080 or other such modules 1091, 1092 (optionally sent upon one or more conditions 1081, 1082 being detected, for example).

In such contexts, one or more sensors 1018, timers 1019, or other modules 1012, 1013, 1014, 1027 of detection logic 1020, control logic 1025, or other implementation logic 1010 may be configured to capture or otherwise obtain one or more invocation times 1011 or other operational parameters 1023, for example, from wireless control signal content 1024. In some variants, for example, such implementation logic selectively accepts one or more components of signals 1031 from one or more modules 1032 of a receiver 1030.

In response one or more dispensers 1051, 1052 may administer or otherwise dispense one or more instances of stimulants 1041, methylxanthines 1042, narcotics 1043, nitric oxide sources 1044, sympathomimetic amines 1045, sedatives 1046 by activating one or more elements 1047. Alternatively or additionally, one or more dispensers 1052, 1053 may schedule one or more times 1056 for dispensations or other events 1057 by receiving records 1055 thereof locally. Alternatively or additionally, one or more dispensers 1051, 1053 may be configured dispense any of two or more available doses 1061, 1062 of a bioactive material, such as by implementing a selected activation modes or respective element 1047 corresponding to a target dosage.

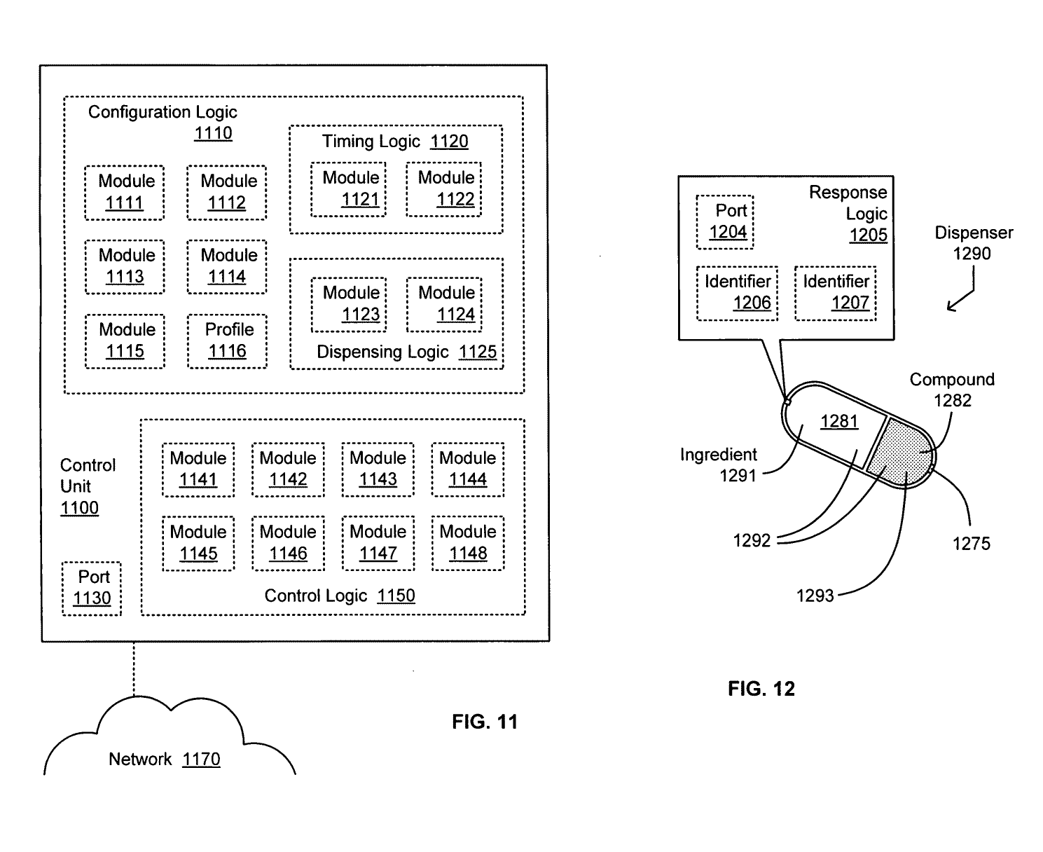

With reference now to FIG. 11, shown is a control unit 1100 operable for interacting (with a local module, e.g.) via one or more instances of ports 1130 and/or networks 1170. In some variants, control unit 1100 may include one or more modules 1111, 1112, 1113, 1114, 1115, 1116, 1121, 1122, 1123, 1124 of timing logic 1120, dispensing logic 1125, or other configuration logic 1110 as described herein. Alternatively or additionally, control unit 1100 may include one or more modules 1141, 1142, 1143, 1144, 1145, 1146, 1147, 1148 of control logic 1150.

With reference now to FIG. 12, shown is a dispenser 1290 operable for responding, such as by dispensing a selected compound 1281, 1282 containing one or more ingredients 1291, 1292, 1293 responsive to control unit 1100 or other such logic. In some variants, for example, at least some such compounds 1281, 1282 are selectively releasable via an addressable valve or other actuator(s) 1275 responsive, for example, to a wireless activation signal. In some variants, for example, response logic 1205 is configured to receive such signals (via one or more local ports 1204, e.g.); to determine which identifier(s) 1206, 1207 (of compounds or reservoirs, e.g.) are present in the signal; and to dispense a subset of such compounds 1281 to administer, accordingly.

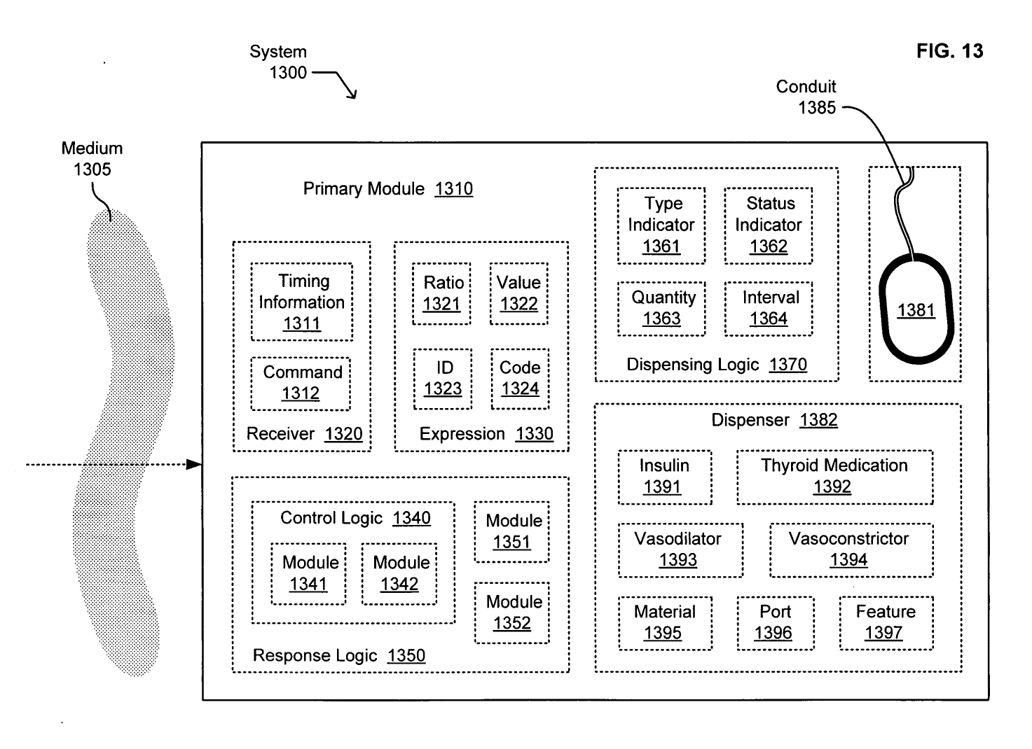

With reference now to FIG. 13, shown is a system 1300 comprising a primary module 1310 operable to receive signals or otherwise to detect physical phenomena through a wireless medium 1305. Primary module 1310 may include receivers 1320 operable for handling one or more instances of timing information 1311, commands 1312, ratios, 1321, values 1322, identifiers 1323, codes 1324, or other expressions 1330 as described herein. Such objects can then be provided, as described below, to one or more modules 1341, 1342, 1351, 1352 of control logic 1340 or other response logic 1350. Alternatively or additionally, one or more type indicators 1361, status indicators 1362, quantities 1363, or intervals 1364 relating to dispensing may be generated or otherwise handled by dispensing logic 1370, such as for controlling one or more dispensers 1381, 1382. In some variants, such dispensers 1381, 1382 may include catheters or other such conduits 1385. Alternatively or additionally, dispensing logic 1370 may cause such dispensers 1381, 1382 to dispense one or more of insulin 1391, a thyroid medication 1392, a vasodilator 1393, a vasoconstrictor 1394, or other materials 1395 via one or more ports 1396 by controlling one or more valves, buffer layers, chemical activators, or other such control features 1397.

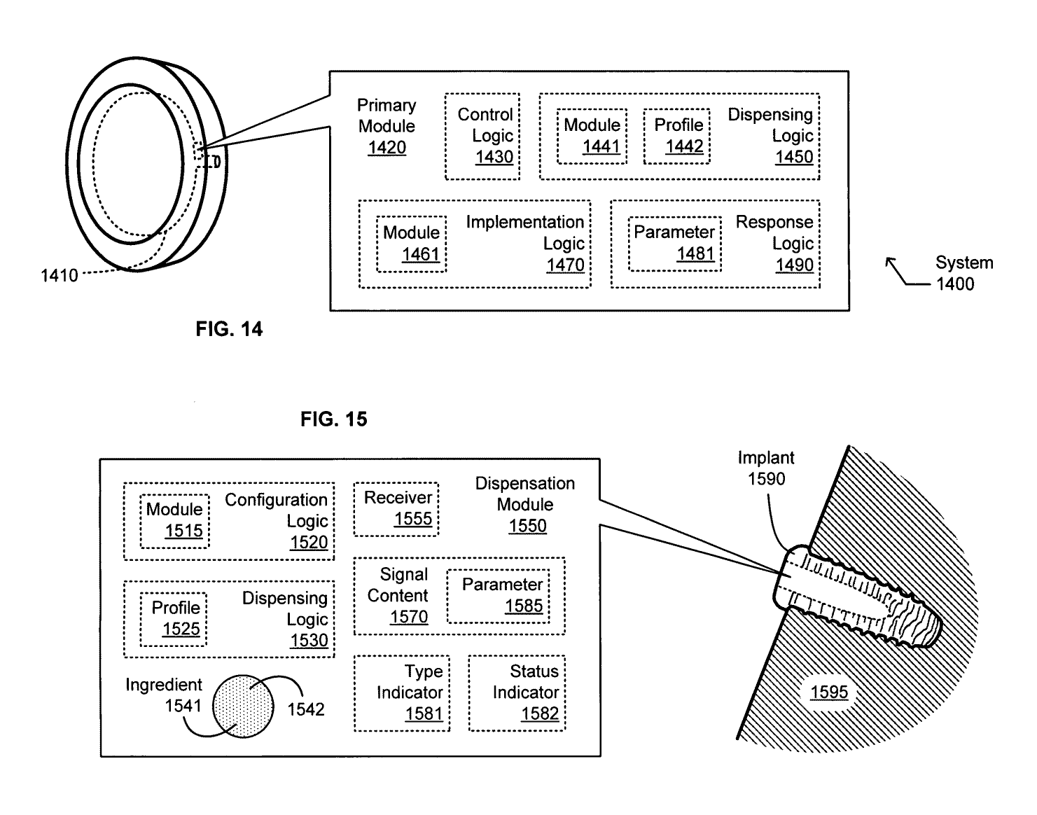

With reference now to FIG. 14, shown is a system 1400 comprising an ingestible or other primary module 1420 operable to control outflow from one or more dispensers 1410 thereof. In some variants, primary module 1420 may include one or more instances of control logic 1430, one or more modules 1441 of dispensing logic, and/or one or more modules 1461 of implementation logic 1470. In some variants, for example, such dispensing logic 1450 or other response logic 1490 may implement one or more profiles 1442 or other parameters 1481 as described below.

With reference now to FIG. 15, shown is a controllable dispensation module 1550 implemented in an implant 1590 in situ (into bone 1595, e.g.). Dispensation module 1550 may include one or more instances of modules 1515 of configuration logic 1520; profiles 1525 of dispensing logic 1530; ingredients 1541, 1542; or receivers 1555 (for extracting parameters 1585 from signal content 1570, e.g.). Alternatively or additionally, such logic may be configured to generate and/or respond to one or more type indicators 1581 or status indicators 1582 as described below.

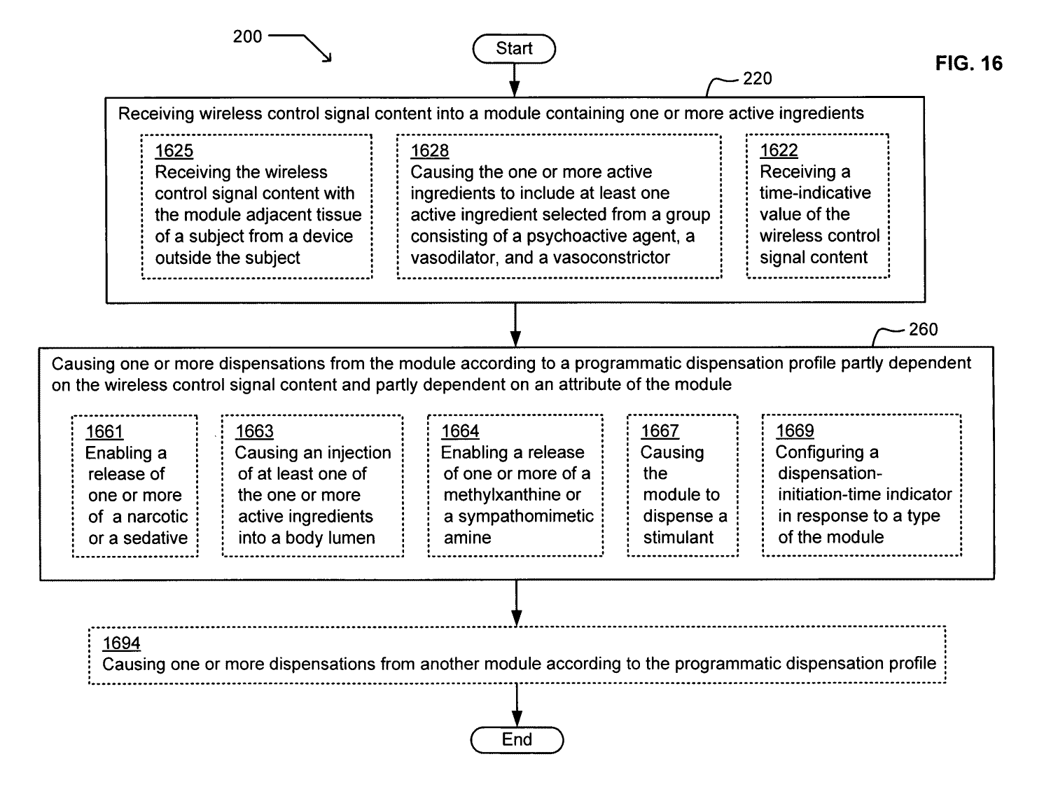

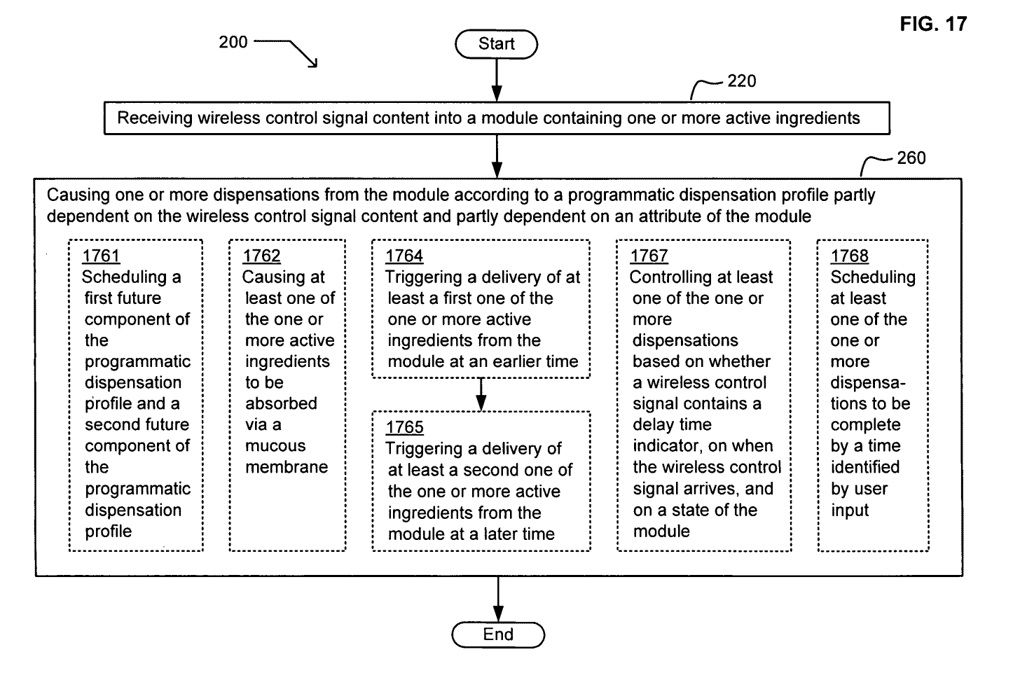

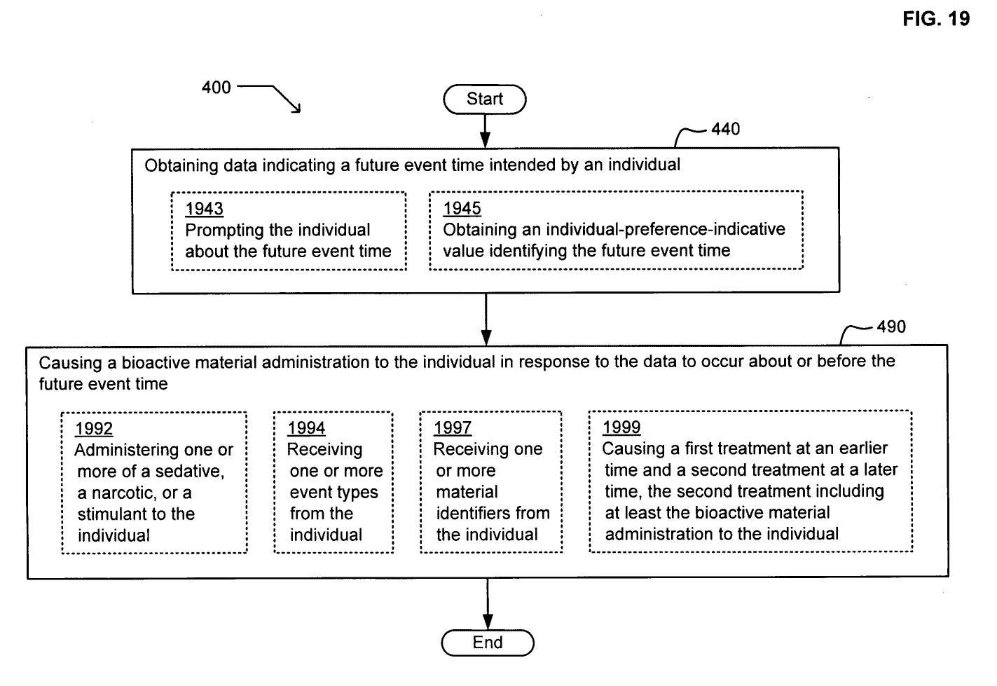

With reference now to FIG. 16, there are shown several variants of the flow 200 of FIG. 2. Operation 220—receiving wireless control signal content into a module containing one or more active ingredients—may (optionally) include one or more of the following operations: 1622, 1625, or 1628. In some embodiments, variants of operation 220 may be performed by one or more instances of receivers 730, 840, 1320; recognition logic 660 or other processing logic 585; control logic 1025, 1340; or the like as exemplified herein. Operation 260—causing one or more dispensations from the module according to a programmatic dispensation profile partly dependent on the wireless control signal content and partly dependent on an attribute of the module—may include one or more of the following operations: 1661, 1663, 1664, 1667, or 1669. In some embodiments, variants of operation 260 may be performed by one or more instances of control logic 830, 1150; dispensing logic 1125, 1370; implementation logic 1010, 1470; or the like as described herein. In some variants, flow 200 may include operation 1694 and/or other operations described, for example, below with reference to FIGS. 17-19.

Operation 1622 describes receiving a time-indicative value of the wireless control signal content (e.g. module 721 of receiver 730 accepting a selection 121, delay value 122, date 123, or other such temporal expressions in an executable command 745 or other such object 141 received across one or more free space media 105). This can occur, for example, in a context in which receiver 730 and one or more modules 762 of timing logic 770 jointly perform operation 220 and in which the transmission time, reception time, initiation time, completion time and/or other appropriate time-indicative value is contained within or added to content 142 transmitted as control signal 161. In some variants, for example, the initiation time for a device action may be contained within one or more command messages to enable a system lag in command processing to be measured. Alternatively or additionally, time indicative data within messages may allow a receiving system to adjust for the potential of messages being received out of order.

Operation 1625 describes receiving the wireless control signal content with the module adjacent tissue of a subject from a device outside the subject (e.g. module 583 of processing logic 585 receiving one or more messages 501, 502 to facilitate a derivation of one or more data streams 561, instructions 562, configuration items 563, or other such content 560 therefrom). This can occur, for example, in a context in which one or more pills 521, patches 522 or other such devices 520 contain processing logic 585 configured to perform operation 220 and in which one or more modules 551 of configuration logic 550 may extract or otherwise generate such content at least partly from such messages. In some variants, for example, the message content may include commands for the device 520 to return status data 571 about a dispenser 540 or power supply, to deliver one or more medicants 532, and/or to enable or disable detection logic 575. Alternatively or additionally, received content may include or otherwise indicate one or more delay times 566, sensor measurement frequencies 567, delivery amounts and rates, threshold adjustment values 568, or other such operational parameters 570.

Operation 1628 describes causing the one or more active ingredients to include at least one active ingredient selected from a group consisting of a psychoactive agent, a vasodilator, and a vasoconstrictor (e.g. module 1341 of control logic 1340 configuring or implementing one or more of a ratio 1321, a material or reservoir identifier 1323, an increment count or other material-quantity-indicative value 1322, a composition-specific code 1324, or other such expression 1330 signifying a type and/or quantity of one or more stimulants 1041, methylxanthines 1042, narcotics 1043, sedatives 1046, vasodilators 1393, vasoconstrictors 1394, compounds including such ingredients, or other such medicants 532). This can occur, for example, in a context in which control logic 1340 performs operation 220 and in which one or more dispensers 1381, 1382 contain such medicants. In some variants, for example, one or more timing events, sensor based events, subject requests, and/or caregiver requests will invoke such control logic 1340 to select one or more active ingredients from a set of compounds available within and/or externally to the delivery system.

Operation 1661 describes enabling a release of one or more of a narcotic or a sedative (e.g. module 1114 of configuration logic 1110 directly or indirectly invoking one or more motors 951, pressure gradients, actuators 952, electric fields 953, and/or other delivery elements 955 to deliver one or more of an available set of compounds containing at least a narcotic 1043 or a sedative 1046). This can occur, for example, in a context in which one or more local modules 950, 1060 include or otherwise interact with one or more or control units 1100 and in which configuration logic 980, 1110 performs operation 260. In some variants, for example, a delivery system may activate one or more pumps to deliver a bioactive ingredient through one or more transdermal needles, catheters, or other conduits 940, 1385. Alternatively or additionally, other delivery methods such as one or more active ingredients configured as a gaseous substance, mist, and/or fine powder may be delivered via inhalation.

Operation 1663 describes causing an injection of at least one of the one or more active ingredients into a body lumen (e.g. module 1352 of response logic 1350 invoking one or more local modules 950, 750, 1060 to release a bioactive material through, toward, or near a body lumen via a port 1204, membrane, catheter, aspirator, or other delivery element 955, 1047). This can occur, for example, in a context in which response logic 1350 performs operation 260 individually and/or in concert with dispensing logic 970 or mechanical components as described herein. In some variants, for example, one or more active ingredients may be introduced into an intravenous delivery system via a port 1396 of an IV tube. Alternatively or additionally, other delivery methods may be implemented as described herein, such as by transdermal injection, implanted devices, catheters, diffusion, absorption, iontophoresis, or other such modalities.

Operation 1664 describes enabling a release of one or more of a methylxanthine or a sympathomimetic amine (e.g. one or more modules 1148 of control logic 1150 scheduling or otherwise causing one or more delivery elements 1047 to release one or more doses 1061, 1062 containing a methylxanthine 1042 and/or sympathomimetic amine 1045). This can occur, for example, in a context in which local module 1060 or authorization module 1090 includes one or more instances of control logic 1150 configured to perform operation 260 and in which any triggering or other criteria 1073 required for authorizing such release have been or may yet be met. In some variants, for example, local module 1060 may include one or more pumps or other elements 1047 operable to inject or otherwise administer at least one such active ingredient into a vasculature or via a subject's other body lumens. In a mist or powder form, for example, such materials may be inhaled or otherwise absorbed via mucous membranes. Alternatively or additionally, the wireless control signal content 1024 may inform which bioactive materials are to be administered; how fast; from which dispensers 1051, 1052, 1053; at what doses 1061, 1062; or by what other delivery parameters 1023.

Operation 1667 describes causing the module to dispense a stimulant (e.g. module 1124 of dispensing logic 1125 invoking one or more motors 951 or other actuators 952, electric fields 953, and/or other delivery elements 955 to deliver one or more of an available set of stimulant-containing compounds 1281). This can occur, for example, in a context in which dispensing logic 1125 performs operation 260 by causing dispenser 1051 to deliver caffeine and/or other bioactive materials containing stimulant 1041 to at least one target subject or region. In some variants, for example, a pump or other actuator 952 may deliver at least the active ingredient(s) through one or more transdermal needles, transvascular catheters, or other such or conduits. Alternatively or additionally, other delivery methods such as one or more active ingredients configured as a gaseous substance, mist, and/or fine powder may be delivered via inhalation.

Operation 1669 describes configuring a dispensation-initiation-time indicator in response to a type of the module (e.g. one or more modules 1012 of implementation logic 1010 designating an invocation time 1011 according to which one or more dispensers 1051, 1052, 1053 are to be used). This can occur, for example, in a context in which implementation logic 1010 performs or otherwise facilitates operation 260, and in which one or more modules 1115 of configuration logic 1110 schedules or enables a dispensation in response to a presence of a particular module or a type identifier 1206 of one or more dispensers 1051, 1052, 1053. Alternatively or additionally, in some variants, the inclusion of an active ingredient in a delivery system may cause a scheduled delivery in anticipation of activity, rest, eating, and/or other such foreseeable and/or detectable activities.

Operation 1694 describes causing one or more dispensations from another module according to the programmatic dispensation profile (e.g. one or more modules 1027 of control logic 1025 triggering a dispensation of one or more nitric oxide sources 1044, compounds 1282, thyroid medications 1392, or other ingredients 1541 from an implant 1590 or other local module upon one or more timed delivery profiles and/or feedback response systems). This can occur, for example, in a context in which control logic 1150 performs operation 260 and in which internal and/or acquired information provides input into the timing and extent of action. In some variants, for example, one or more delivery systems may be invoked to deliver at least one active ingredient based upon a scheduled delivery time. Alternatively or additionally, module 1027 may be configured to invoke one or more delivery systems based upon a sensor response to an active ingredient level in a detection region, to an observable physiological response, or to one or more measurement thresholds or other such criteria as described herein.

With reference now to FIG. 16, there are shown several variants of the flow 200 of FIG. 2 or 16. Operation 260—causing one or more dispensations from the module according to a programmatic dispensation profile partly dependent on the wireless control signal content and partly dependent on an attribute of the module—may include one or more of the following operations: 1761, 1762, 1764, 1765, 1767, or 1768. In some embodiments, variants of operation 260 may be performed by one or more instances of response logic 1350, 1490; control logic 830, 1150; dispensing logic 1125, 1370, 1450; implementation logic 1010, 1470; timing logic 770, 1120; or the like as described herein.

Operation 1761 describes scheduling a first future component of the programmatic dispensation profile and a second future component of the programmatic dispensation profile (e.g. module 1122 of timing logic 1120 implementing records 1055 specifying at least two times 1056 at which respective events 1057 are to occur). This can occur, for example, in a context in which local module 1060 embodies or implements control unit 1100, in which configuration logic 1110 performs operation 260 and in which one or more dispensers 1051, 1052, 1053 implement profile 1116. In some contexts, for example, adsorption and metabolism rate indicators for a subject are available for specifying an improved dosing profile for an active ingredient. In some variants, module 1122 may be configured to use such indicators to create a continual dosing profile suitable for maintaining a sufficient level of the active ingredient.

Operation 1762 describes causing at least one of the one or more active ingredients to be absorbed via a mucous membrane (e.g. module 981 of configuration logic 980 invoking an administration of one or more such ingredients 941 through a respiratory, digestive, or other such body lumen). This can occur, for example, in a context in which dispensing logic 1125 performs operation 260 and in which the active ingredient is in a form that may be absorbed through a mucous membrane 945. In some variants, for example, gaseous, liquid, and/or fine powder compounds (containing insulin, e.g.) may be inhaled and adsorbed by lung tissue. Alternatively or additionally, an oral cavity or sinus passage may likewise be used for an effective delivery of many therapeutic compounds.

Operation 1764 describes triggering a delivery of at least a first one of the one or more active ingredients from the module at an earlier time (e.g. module 1145 of control logic 1150 signaling a delivery of a sedative or other such ingredients from one or more reservoirs 190 of module 170). This can occur, for example, in a context in which one or more modules 1121 of timing logic 1120 and other control logic 1150 jointly perform operation 260 and in which it is desirable to deliver one or more active ingredients before a set time and/or event. In some variants, for example, delivery of an active ingredient may be based upon subject-specific events such as awakening, falling asleep, meetings, meal times, performances, or other such expected or other future events. Alternatively or additionally, in light of these teachings, delivery of two or more active ingredients (scheduled at different times, e.g.) may be configured to increase effectiveness and/or decrease side effects that might otherwise occur.

Operation 1765 describes triggering a delivery of at least a second one of the one or more active ingredients from the module at a later time (e.g. module 1146 of control logic 1150 signaling a delivery of a stimulant of other such ingredients from one or more reservoirs 190 of module 170). This can occur, for example, in a context in which one or more modules 1121 of timing logic 1120 and other control logic 1150 jointly perform operation 260 and in which it is desirable to deliver one or more active ingredients after the prior dispensation(s). Alternatively or additionally, delivery of one or more active ingredients in succession may increase effectiveness and/or decrease side effects.

Operation 1767 describes controlling at least one of the one or more dispensations based on whether a wireless control signal contains a delay time indicator, on when the wireless control signal arrives, and on a state of the module (e.g. module 1342 of control logic 1340 triggering a quantity 1363 of a material of type indicator 1361 for one or more dispensers 1381, 1382 to administer at least interval 1364 after primary module 1310 receives command 1312 across medium 1305). This can occur, for example, in a context in which control logic 1340 performs operation 260 and in which control signals arriving via receiver 1320 contain initiation, transmission, delay, and/or other timing information 1311. In some variants, for example, delivery timing and volume issues due to transmission delays from remote sites, interference, or signal interruption may be adjusted for.

Operation 1768 describes scheduling at least one of the one or more dispensations to be complete by a time identified by user input (e.g. module 761 of timing logic 770 invoking user interface 705 by which one or more subjects or caregivers may schedule a delivery of one or more active ingredients 1291, 1292 to occur upon or before a stated time or other detected condition 794). This can occur, for example, in a context in which timing logic 770 and/or one or more user interfaces 705 perform operation 260 and in which such input 707 is desirable for selection of delivery module timing. Alternatively or additionally, one or more modules 715 of decision logic 720 may be configured to establish or update one or more delivery-time-indicative tasks 795 in response to one or more future meals, discomfort levels, device interactions, detectable user actions, or other such foreseeable conditions.

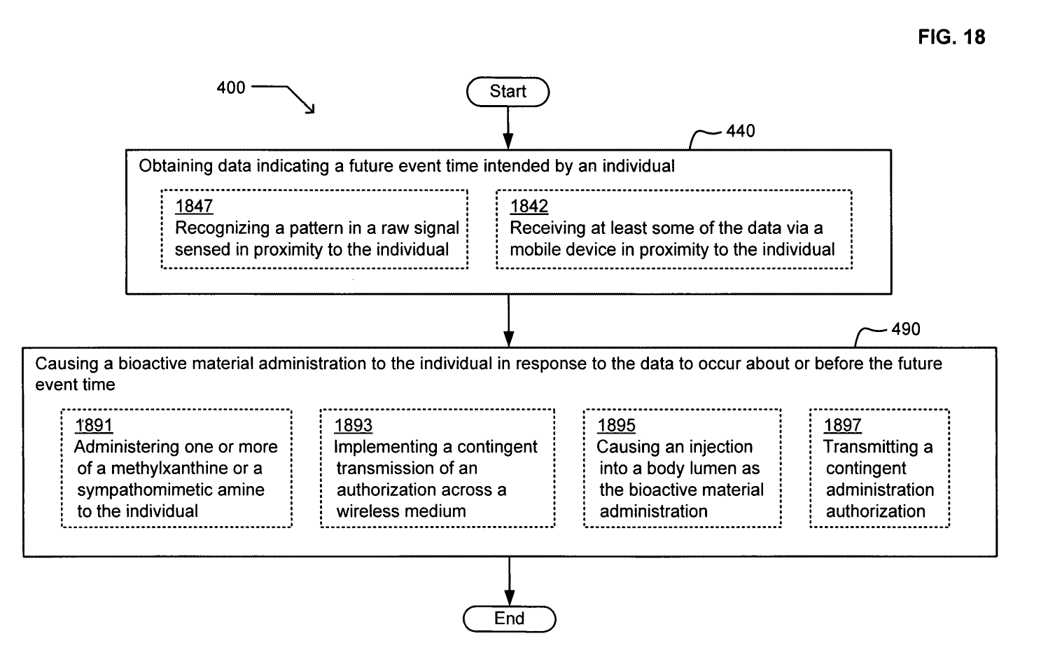

With reference now to FIG. 18, there are shown several variants of the flow 400 of FIG. 4. Operation 440—obtaining data indicating a future event time intended by an individual—may (optionally) include one or more of the following operations: 1842 or 1847. In some embodiments, variants of operation 440 may be performed by one or more instances of receivers 730, 960, 1555; recognition logic 660; or other response logic 590, 1205, 1350. Operation 490—causing a bioactive material administration to the individual in response to the data to occur about or before the future event time—may include one or more of the following operations: 1891, 1893, 1895, or 1897. In some embodiments, variants of operation 490 may be performed by one or more instances of scheduling logic 350; response logic 760, 1205; authorization logic 1090; dispensing logic 780, 1450, 1530; configuration logic 810, 980, 1520; or the like as described herein.

Operation 1842 describes receiving at least some of the data via a mobile device in proximity to the individual (e.g. module 1351 of response logic 1350 receiving input 707 including an upcoming event description, a dispensation-indicative expression 1330, or other such information via a watch, cell phone, implant, or other portable device 708). This can occur, for example, in a context in which primary module 1310 receives within or via local module 750 and in which one or more receivers 730, 1320 and response logic 1350 jointly perform operation 440. In some variants, for example, one or more individuals may provide such input 707 in preparation for a specified event, permitting response logic 1350 to configure one or more dispensations locally to the individual (via an implant, for example, or a patch or capsule containing a medicant). Alternatively or additionally, one or more modules 654 of recognition logic 660 may be configured to receive and recognize patterns in raw or other data, for example, received via one or more sensors 623, 624.

Operation 1847 describes recognizing a pattern in a raw signal sensed in proximity to the individual (e.g. module 651 of recognition logic 660 seeking one or more patterns 658 indicative of a future event time 631 or other event information 630 in raw data 643 from one or more auditory sensors 621 or other sensors 624 local to user 610). This can occur, for example, in a context in which at least recognition logic 660 performs operation 440 and in which one or more modules 652, 653 of recognition logic 660 are applied to auditory or optical data. In some variants, for example, a voice recognition module 652 may be used (a) to recognize one or more spoken instructions and/or (b) to confirm a subject's identity before performing the instruction(s). Alternatively or additionally, patterns 657 in temperature, heart rate, skin coloration, blood pressure, subject movement, fluid excretion, historical data, and/or other data 644 about user 610 may be used as a decision point for required and/or desired interaction by an device and/or caregiver. Alternatively or additionally, one or more other modules 654 of recognition logic 660 may be configured to perform other aspects and/or variants of operation 440 as described herein.

Operation 1891 describes administering one or more of a methylxanthine or a sympathomimetic amine to the individual (e.g. module 753 of response logic 760 activating one or more elements 1047 for dispensing one or more methylxanthines 1042, sympathomimetic amines 1045, and/or other ingredients 741 from dispenser 740). This can occur, for example, in a context in which dispensing logic 780 and one or more dispensers 740 jointly perform operation 490 by delivering one or more substances to the subject and in which the substances come from a set containing at least methylxanthines and/or sympathomimetic amines.

Operation 1893 describes implementing a contingent transmission of an authorization across a wireless medium (e.g. module 1092 of authorization logic 1090 transmitting one or more authorizations 1071, 1072 through air, body tissue, or other such media 1065 to local module 1032 contingent upon one or more conditions 1082). This can occur, for example, in a context in which implementation logic 1010 and/or authorization logic 1090 are configured perform operation 490, in which control logic 1080 is configured to detect one or more loop conditions or other such conditions 1082 frequently, and in which such contingent transmission are effective for saving significant system resources. In some variants, for example, such contingent transmissions may save battery power or processing resources, for example, within local module 1060 or other system components. Alternatively or additionally, in some contexts, such contingent transmissions may be effective for minimizing noise, interference, or other such undesirable consequences.

Operation 1895 describes causing an injection into a body lumen as the bioactive material administration (e.g. module 781 of dispensing logic 780 triggering one or more dispensers 740 to inject one or more ingredients 741, 742 that include a bioactive material into one or more subject body lumens). This can occur, for example, in a context in which dispensing logic 780 and the dispenser(s) 740 jointly perform operation 490 by delivering one or more of insulin 1391, a thyroid medication 1392, or other materials 1395 prescribed in relation to a future event by a physician via a mucous membrane, a body lumen, or otherwise by injection. In some variants, for example, delivery of a substance to the subject can occur using a needle, catheter, or other type of port 1396 to establish a direct path to the target site. Alternatively or additionally, one or more dispensers 1381, 1382 may be configured with one or more features 1397 to facilitate transdermal absorption, iontophoresis, transmucosal adsorption, or other indirect modes of administration to the subject.

Operation 1897 describes transmitting a contingent administration authorization (e.g. module 1091 of authorization logic 1090 transmitting an authorization 1072 to local module 1032 that will not take effect unless one or more criteria 1073 are met). This can occur, for example, in a context in which authorization logic 1090 and implementation logic 1010 jointly and/or successively perform operation 490 and in which one or more modules 1013 of implementation logic 1010 are configured to monitor a sensor 1018, timer 1019, or other detection logic 1020 to determine whether any such criteria are met. In some variants, for example, control logic 1080 triggers such a transmission when a physician or other human being requests a contingent administration, or when one or more other such conditions 1081 are met. Alternatively or additionally, other components of authorization logic 1090 may transmit an override or other such authorization 1071 as described herein.

With reference now to FIG. 18, there are shown several variants of the flow 400 of FIG. 4. Operation 440—obtaining data indicating a future event time intended by an individual—may (optionally) include one or more of the following operations: 1842 or 1847. In some embodiments, variants of operation 440 may be performed by one or more instances of receivers 730, 960, 1555 or other recognition logic 660; response logic 590, 1205, 1350; or the like as described herein. Operation 490—causing a bioactive material administration to the individual in response to the data to occur about or before the future event time—may include one or more of the following operations: 1891, 1893, 1895, or 1897. In some embodiments, variants of operation 490 may be performed by one or more instances or combinations of scheduling logic 350; response logic 760, 1205; authorization logic 1090; dispensing logic 780, 1530; configuration logic 810, 1520; or the like as described herein.

Operation 1943 describes prompting the individual about the future event time (e.g. module 754 of response logic 760 receiving input 707 via interface 705 confirming or otherwise indicating event information 630 after presenting output 706 to a user 610 or other individual 710 to whom an administration is possible). This can occur, for example, in a context in which response logic 760 performs operation 440 via one or more instances of interface 705 in a vicinity 613 of user 610. In some variants, for example, one or more modules 654 of recognition logic 660 may recognize one or more data patterns 658 indicative of an apparent habit, an indication from a caregiver, a schedule, or other such indirect sources. In such contexts, module 754 of response logic 760 may confirm such timing or other event information 630 by querying user 610 about such events. Alternatively or additionally, the presumptive timing or other prompting may be presented to user 610 via a cell phone or other portable device.

Operation 1945 describes obtaining an individual-preference-indicative value identifying the future event time (e.g. module 722 of receiver 730 receiving one or more identifiers 725 of such preferences as input 707 from individual 710). This can occur, for example, in a context in which receiver 730 performs operation 490 directly. Alternatively or additionally, one or more modules 763 of timing logic 770 may be configured to extract one or more estimates of future event times 631, event types 632, and/or other such event information 630 in response to the individual's habits, scheduling information, or other such indirect indications. In some contexts, moreover, such inferences may be subject to confirmation by interacting with the individual and/or a caregiver.

Operation 1992 describes administering one or more of a sedative, a narcotic, or a stimulant to the individual (e.g. module 752 of response logic 760 activating one or more elements 1047 for dispensing one or more stimulants 1041, narcotics 1043, sedatives 1046, and/or other ingredients 742 from dispenser 740). This can occur, for example, in a context in which in which one or more dispensers 740 contain at least one such active ingredient and in which response logic 760 may perform operation 490 by activating one or more motors, actuators. In some variants, for example, a sedative may be selected for administration to a subject prior to a rest time, or as a result of other physical or emotional conditions. Alternatively or additionally, substance combinations may be used to reduce side effects of one of the substances and/or to increase effectiveness of one of the substances.

Operation 1994 describes receiving one or more event types from the individual (e.g. module 723 of receiver 730 obtaining event names or other descriptive event types 632 entered by user 610). This can occur, for example, in a context in which receiver 730 includes or otherwise responds to one or more sensors 624 in a vicinity 613 of user 610. In some variants, for example, one or more individuals 710 may request timing for substance delivery, therapy application, monitoring activation, and/or monitoring deactivation from a user interface 705. Alternatively or additionally, user interface 705 may be configured to project or otherwise present a list of available event types 632 to the individual(s) 710.

Operation 1997 describes receiving one or more material identifiers from the individual (e.g. module 724 of receiver 730 obtaining one or more identifiers 725 of one or more nitric oxide sources 1044 or other ingredients 741, 742 from one or more individuals 710 at user interface 705). This can occur, for example, in a context in which receiver 730 and one or more dispensers 740 jointly perform operation 490 by administering or otherwise dispensing such materials to an individual 710 who identified the ingredient(s). In some variants, for example, delivery modules that are preloaded with a substance may be programmed with the identity and amount of that substance when connected to or activated by one or more modules 751 of response logic 760. Alternatively or additionally, delivery modules containing multiple reservoirs may be configured to identify the substance and/or substance amount in each reservoir. In some variants, for example, device 520 may identify one or more medicants 531 or other ingredients 533 that are in short supply, that are in ample supply, that have been loaded or reloaded, or for which other such status data 571 applies.

Operation 1999 describes causing a first treatment at an earlier time and a second treatment at a later time, the second treatment including at least the bioactive material administration to the individual (e.g. one or more modules 981 of configuration logic 980 implementing a dispensation profile 784 or otherwise causing respective tasks 795 to be performed at respective times 791 such that a latter one of the tasks 795 includes an administration of a stimulant 1041 or other bioactive material). This can occur, for example, in a context in which one or more local modules 950, 750, 1060 include or otherwise interact with one or more dispensers 540, 740, 1051, 1052 operable to perform such composite or otherwise coordinated treatments. In some variants, for example, configuration logic 550, 980, 1520 may invoke the application of a physical treatment, muscle stimulation, organ stimulation, nerve stimulation and/or delivery of a bioactive substance to the subject as an earlier treatment. Alternatively or additionally, a later treatment may include the bioactive material administration, optionally in concert with a second application of a physical treatment, muscle stimulation, organ stimulation, nerve stimulation, or other delivery.

An embodiment provides one or more modules 1112 of configuration logic 1110 configured as circuitry for causing dispenser 1290 to administer a succession of compounds 1281, 1282 according to a profile 1116. This can occur, for example, in a context in which module 1111 configures profile 1116 according to a desired release rate, concentration, composition, activity, remaining amount, or other aspect of one or more compounds 1282 or other ingredients 1293 in a dispenser 1290 or other module. In some contexts, for example, module 1112 may cause an imminent release of ingredient 1293 selectively by addressing actuator 1275. Alternatively or additionally, module 1111 may define a profile calling for such a dispensation in response to receiving one or more attributes of dispenser 1290 in a wireless signal received via network 1170.

Some or all of the embodiments described herein may generally comprise technologies for handling one or more bioactive agents and/or carriers in releasable module form, via a liquid-bearing conduit, in a mist or other spray form, in a pumped or other pressurized form, or otherwise according to technologies described herein. In a general sense, those skilled in the art will recognize that the various aspects described herein which can be implemented, individually and/or collectively, by a wide range of hardware, software, firmware, or any combination thereof can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of random access memory), and/or electrical circuitry forming a communications device (e.g., a modem, communications switch, or optical-electrical equipment). Those having skill in the art will recognize that the subject matter described herein may be implemented in an analog or digital fashion or some combination thereof.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In one embodiment, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a floppy disk, a hard disk drive, a Compact Disc (CD), a Digital Video Disk (DVD), a digital tape, a computer memory, etc.; and a transmission type medium such as a digital and/or an analog communication medium (e.g., a fiber optic cable, a waveguide, a wired communications link, a wireless communication link (e.g., transmitter, receiver, transmission logic, reception logic, etc.), etc.).

All of the above-mentioned U.S. patents, U.S. patent application publications, U.S. patent applications, foreign patents, foreign patent applications and non-patent publications referred to in this specification and/or listed in any Application Data Sheet, are incorporated herein by reference, to the extent not inconsistent herewith.

One skilled in the art will recognize that the herein described components (e.g., operations), devices, objects, and the discussion accompanying them are used as examples for the sake of conceptual clarity and that various configuration modifications are contemplated. Consequently, as used herein, the specific exemplars set forth and the accompanying discussion are intended to be representative of their more general classes. In general, use of any specific exemplar is intended to be representative of its class, and the non-inclusion of specific components (e.g., operations), devices, and objects should not be taken limiting.

With respect to the use of substantially any plural and/or singular terms herein, those having skill in the art can translate from the plural to the singular and/or from the singular to the plural as is appropriate to the context and/or application. The various singular/plural permutations are not expressly set forth herein for sake of clarity.

The herein described subject matter sometimes illustrates different components contained within, or connected with, different other components. It is to be understood that such depicted architectures are merely exemplary, and that in fact many other architectures may be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled,” to each other to achieve the desired functionality, and any two components capable of being so associated can also be viewed as being “operably couplable,” to each other to achieve the desired functionality. Specific examples of operably couplable include but are not limited to physically mateable and/or physically interacting components, and/or wirelessly interactable, and/or wirelessly interacting components, and/or logically interacting, and/or logically interactable components.

In some instances, one or more components may be referred to herein as “configured to,” “configurable to,” “operable/operative to,” “adapted/adaptable,” “able to,” “conformable/conformed to,” etc. Those skilled in the art will recognize that “configured to” can generally encompass active-state components and/or inactive-state components and/or standby-state components, unless context requires otherwise.

While particular aspects of the present subject matter described herein have been shown and described, it will be apparent to those skilled in the art that, based upon the teachings herein, changes and modifications may be made without departing from the subject matter described herein and its broader aspects and, therefore, the appended claims are to encompass within their scope all such changes and modifications as are within the true spirit and scope of the subject matter described herein. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

With respect to the appended claims, those skilled in the art will appreciate that recited operations therein may generally be performed in any order. Also, although various operational flows are presented in a sequence(s), it should be understood that the various operations may be performed in other orders than those which are illustrated, or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

Those skilled in the art will recognize that it is common within the art to implement devices and/or processes and/or systems, and thereafter use engineering and/or other practices to integrate such implemented devices and/or processes and/or systems into more comprehensive devices and/or processes and/or systems. That is, at least a portion of the devices and/or processes and/or systems described herein can be integrated into other devices and/or processes and/or systems via a reasonable amount of experimentation. Those having skill in the art will recognize that examples of such other devices and/or processes and/or systems might include—as appropriate to context and application—all or part of devices and/or processes and/or systems of (a) an air conveyance (e.g., an airplane, rocket, helicopter, etc.), (b) a ground conveyance (e.g., a car, truck, locomotive, tank, armored personnel carrier, etc.), (c) a building (e.g., a home, warehouse, office, etc.), (d) an appliance (e.g., a refrigerator, a washing machine, a dryer, etc.), (e) a communications system (e.g., a networked system, a telephone system, a Voice over IP system, etc.), (f) a business entity (e.g., an Internet Service Provider (ISP) entity such as Comcast Cable, Qwest, Southwestern Bell, etc.), or (g) a wired/wireless services entity (e.g., Sprint, Cingular, Nextel, etc.), etc.

In certain cases, use of a system or method may occur in a territory even if components are located outside the territory. For example, in a distributed computing context, use of a distributed computing system may occur in a territory even though parts of the system may be located outside of the territory (e.g., relay, server, processor, signal-bearing medium, transmitting computer, receiving computer, etc. located outside the territory).

A sale of a system or method may likewise occur in a territory even if components of the system or method are located and/or used outside the territory. Further, implementation of at least part of a system for performing a method in one territory does not preclude use of the system in another territory.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A treatment system comprising:
circuitry for obtaining data indicating a future event time intended by an individual; and
circuitry for causing a bioactive material administration to the individual in response to the data to occur about or before the future event time.

2. The treatment system of claim 1, further comprising:
a dispenser; and
an in-dwelling conduit configured to facilitate the bioactive material administration from the dispenser to the individual.

3. The treatment system of claim 1, further comprising:
a dispenser operable for administering a medicant to the individual responsive to the circuitry for causing the bioactive material administration.

4. The treatment system of claim 1, further comprising:
a dispenser operable for releasing at least a psychoactive material responsive to the circuitry for causing the bioactive material administration to the individual.

5. The treatment system of claim 1, further comprising:
a patch configured to initiate the bioactive material administration to the individual before the future event time.

6. The treatment system of claim 1 in which the circuitry for obtaining data indicating a future event time intended by an individual comprises:
one or more auditory sensors.

7. The treatment system of claim 1 in which the circuitry for obtaining data indicating a future event time intended by an individual comprises:
circuitry for facilitating a transmission through a wireless medium.

8. The treatment system of claim 1 in which the circuitry for obtaining data indicating a future event time intended by an individual comprises:
circuitry for recognizing a pattern in a raw signal sensed in proximity to the individual.

9. The treatment system of claim 1 in which the circuitry for obtaining data indicating a future event time intended by an individual comprises:
circuitry for receiving at least some of the data via a mobile device in proximity to the individual.

10. The treatment system of claim 1 in which the circuitry for obtaining data indicating a future event time intended by an individual comprises:
circuitry for prompting the individual about the future event time.

11. The treatment system of claim 1 in which the circuitry for obtaining data indicating a future event time intended by an individual comprises:
circuitry for obtaining an individual-preference-indicative value identifying the future event time.

12. The treatment system of claim 1 in which the circuitry for causing a bioactive material administration to the individual in response to the data to occur about or before the future event time comprises:
circuitry for causing an administration of one or more of a methylxanthine or a sympathomimetic amine to the individual.

13. The treatment system of claim 1 in which the circuitry for causing a bioactive material administration to the individual in response to the data to occur about or before the future event time comprises:
circuitry for implementing a contingent transmission of an authorization across a wireless medium.

14. The treatment system of claim 1 in which the circuitry for causing a bioactive material administration to the individual in response to the data to occur about or before the future event time comprises:
circuitry for causing an injection into a body lumen as the bioactive material administration.

15. The treatment system of claim 1 in which the circuitry for causing a bioactive material administration to the individual in response to the data to occur about or before the future event time comprises:
circuitry for transmitting a contingent administration authorization.

16. The treatment system of claim 1 in which the circuitry for causing a bioactive material administration to the individual in response to the data to occur about or before the future event time comprises:
circuitry for causing an administration of one or more of a sedative, a narcotic, or a stimulant to the individual.

17. The treatment system of claim 1 in which the circuitry for causing a bioactive material administration to the individual in response to the data to occur about or before the future event time comprises:
circuitry for receiving one or more event types from the individual.

18. The treatment system of claim 1 in which the circuitry for causing a bioactive material administration to the individual in response to the data to occur about or before the future event time comprises:
circuitry for receiving one or more material identifiers from the individual.

19. The treatment system of claim 1 in which the circuitry for causing a bioactive material administration to the individual in response to the data to occur about or before the future event time comprises:
circuitry for causing a first treatment at an earlier time and a second treatment at a later time, the second treatment including at least the bioactive material administration to the individual.

20. A treatment system comprising:
means for obtaining data indicating a future event time intended by an individual; and
means for causing a bioactive material administration to the individual in response to the data to occur about or before the future event time.

21-24. (canceled)

25. The treatment system of claim 20 in which the means for causing a bioactive material administration to the individual in response to the data to occur about or before the future event time comprises:
means for administering one or more of a methylxanthine or a sympathomimetic amine to the individual.

26-29. (canceled)

30. The treatment system of claim 20 in which the means for causing a bioactive material administration to the individual in response to the data to occur about or before the future event time comprises:
means for receiving one or more event types from the individual.

31. The treatment system of claim 20 in which the means for causing a bioactive material administration to the individual in response to the data to occur about or before the future event time comprises:
means for receiving one or more material identifiers from the individual.

32. (canceled)

33. A treatment method comprising:
obtaining data indicating a future event time intended by an individual; and
causing a bioactive material administration to the individual in response to the data to occur about or before the future event time.

34-40. (canceled)

41. The treatment method of claim 33 in which the causing a bioactive material administration to the individual in response to the data to occur about or before the future event time comprises:
transmitting a contingent administration authorization.

42-44. (canceled)

45. The treatment method of claim 33 in which the causing a bioactive material administration to the individual in response to the data to occur about or before the future event time comprises:
causing a first treatment at an earlier time and a second treatment at a later time, the second treatment including at least the bioactive material administration to the individual.

46. The treatment system of claim 1, further comprising:
a dispenser operable for administering a medicant to the individual responsive to the circuitry for causing the bioactive material administration; and
an in-dwelling conduit configured to facilitate the bioactive material administration from the dispenser to the individual.

47. The treatment system of claim 1 in which the circuitry for obtaining data indicating a future event time intended by an individual comprises:
circuitry for recognizing a pattern in a raw signal sensed in proximity to the individual;
circuitry for receiving at least some of the data via a mobile device in proximity to the individual;
circuitry for facilitating a transmission through a wireless medium;
circuitry for prompting the individual about the future event time; and
circuitry for obtaining an individual-preference-indicative value identifying the future event time.

48. The treatment system of claim 47 in which the circuitry for causing a bioactive material administration to the individual in response to the data to occur about or before the future event time comprises:
circuitry for transmitting a contingent administration authorization;
circuitry for causing a contingent injection into a body lumen as the bioactive material administration.

49. The treatment system of claim 1 in which the circuitry for causing a bioactive material administration to the individual in response to the data to occur about or before the future event time comprises:
circuitry for transmitting a contingent administration authorization;
circuitry for causing a contingent injection into a body lumen as the bioactive material administration.

50. The treatment system of claim 1 in which the circuitry for causing a bioactive material administration to the individual in response to the data to occur about or before the future event time comprises:
circuitry for receiving one or more material identifiers from the individual;
circuitry for receiving one or more event types from the individual;
circuitry for causing an injection into a body lumen as the bioactive material administration; and
circuitry for causing a first treatment at an earlier time and a second treatment at a later time, the second treatment including at least the bioactive material administration to the individual.
